(19) United States
(12) Patent Application Publication
(10) Pub. No.: US 2022/0189275 A1
(43) Pub. Date: Jun. 16, 2022

(54) PATIENT ALERT AND MONITORING SYSTEM

(71) Applicant: Patricia E. Wilson, Parkville, MD (US)

(72) Inventor: Patricia E. Wilson, Parkville, MD (US)

(21) Appl. No.: 17/543,874

(22) Filed: Dec. 7, 2021

Related U.S. Application Data
(60) Provisional application No. 63/124,131, filed on Dec. 11, 2020.

(51) Int. Cl. G08B 21/02; A61B 5/00; G08B 21/18

(52) U.S. Cl. CPC G08B 21/02; G08B 21/182; A61B 5/7405; A61B 5/746

(57) ABSTRACT

A patient alert and monitoring system for use in a hospital, such as, an ICU setting, in which each patient, area, or group is provided with a monitoring system and a distinctive individual alert associated with a particular health care worker. The health care worker, such as a nurse, has a monitor which is connected to the monitoring systems. When a patient requires attention, the distinctive alert associated with that patient is activated on the health care worker's monitor, whereby the worker will be alerted to the particular patient in need of attention without requiring the worker to be at a main or central station. Since each health care worker has its own distinctive alert, other health care workers will know that the alert or alert does not concern them and will not be distracted. A standard alert is included and activated as a back-up to the individual worker alerts.

### CROSS REFERENCE TO RELATED APPLICATIONS

This application claims priority to U.S. Provisional Patent Application Ser. No. 63/124,131 filed Dec. 11, 2020, the disclosure of which is incorporated herein by reference in its entirety.

### BACKGROUND OF THE INVENTION

### 1. Field of the Invention

The present invention relates, in general, to heal care, and more particularly, to patient monitoring in a hospital.

### 2. Brief Description of the Prior Art

Hospitals, nursing homes, and other patient care facilities typically include patient monitoring devices at one or more bed sides in the facility. Patient monitoring devices generally include sensors, processing equipment, and displays for obtaining and analyzing a medical patient's physiological parameters, such as, blood oxygen saturation level, respiratory rate, and the like. Clinicians, including doctors, nurses, and other medical personnel, use the physiological parameters obtained from patient monitors to diagnose illnesses and to prescribe treatments. Clinicians also use the physiological parameters to monitor patients during various clinical situations to determine whether to increase the level of medical care given to patients.

For example, the patient monitoring devices can be used to monitor a pulse oximeter. Pulse oximetry is a widely accepted non-invasive procedure for measuring the oxygen saturation level of arterial blood, which is an indicator of a person's oxygen supply. A typical pulse oximetry system utilizes an optical sensor clipped onto a fingertip to measure the relative volume of oxygenated hemoglobin in pulsatile arterial blood flowing within the fingertip. Oxygen saturation (SpO2), pulse rate, a plethysmograph waveform, perfusion index (PI), pleth variability index (PVI), methemoglobin (MetHb), carboxyhemoglobin (CoHb), total hemoglobin (tHb), and glucose can be displayed on a monitor, accordingly.

The patient monitoring devices can also communicate with an acoustic sensor comprising an acoustic transducer, such as a piezoelectric element. The acoustic sensor can detect respiratory and other biological sounds of a patient and provide signals reflecting these sounds to a patient monitor.

Blood pressure is another example of a physiological parameter that can be monitored. Many devices allow blood pressure to be measured by sphygmomanometer systems that utilize an inflatable cuff applied to a person's arm. The cuff is inflated to a pressure level high enough to occlude a major artery. When air is slowly released from the cuff, blood pressure can be estimated by detecting “Korotkoff” sounds using a stethoscope or other detection means placed over the artery. Other examples of physiological parameters which can be measured include respiration rate, blood analyte measurements, such as oxygen saturation, and ECG.

In the past, these monitors have been connected to a readout unit located at a nursing station. A nurse at the station is alerted as necessary by the patient monitors and takes appropriate action. However, this requires the nurse to be physically located at the nursing station. This is not always convenient or practical since nurses are often required to be at various locations throughout a hospital.

As discussed herein above, devices for measuring various physiological parameters, or “vital signs,” of a patient; such as, temperature, blood pressure, heart rate, heart activity, etc., have been a standard part of medical care for many years. Indeed, the vital signs of some patients (e.g., those undergoing relatively moderate to high levels of care) typically are measured on a substantially continuous basis to enable physicians, nurses and other health care providers to detect sudden changes in a patient's condition and evaluate a patient's condition over an extended period of time. However, for most hospitals and other medical facilities care for numerous patients assigned to numerous different rooms, it can be difficult for a finite number of clinicians to monitor multiple patients on a continuous basis. In an effort to alleviate this problem, some medical monitoring systems have been developed to enable the vital signs data collected from patients to be conveyed to a central location, thereby allowing one or a few clinicians to simultaneously monitor multiple patients in different locations.

There is, therefore, a need in the art to provide a means and system to monitor patients in a manner that can alert a nurse or other health care workers to a patient's need without requiring the health care worker to be physically located at a central station, such as a nursing station.

### SUMMARY OF THE INVENTION

The present invention meets this need. The present invention relates to a patient alert and monitoring system. A first embodiment of the present invention provides a patient alert system for use in a hospital, particularly for an ICU setting, in which each patient or area has a monitoring system with an individual alert. A unique sound is assigned to each health care worker, such as a nurse, and that health care worker has a monitor which is connected to the patient-monitoring systems. When a particular patient requires attention, the distinctive alert associated with the health care worker associated with that patient is activated on the health care worker's monitor, whereby the worker will be alerted to the particular patient in need of attention without the health care worker being required to be at a main or central station. The unique nature of the alert or alert obviates the possibility that other health care workers are distracted by the alarm.

A second embodiment of the invention provides a wireless patient monitor which is adapted to communicate with any one of several medical telemetry networks, each having one or more central stations, where each network is configured to communicate via wireless communications using one of a multiple of different communications settings. The patient monitor can include one or more sensor inputs for receiving vital signs data from a patient, a wireless transceiver configured to transmit and receive wireless communications, and a controller coupled to receive the vital signs data via one or more sensor inputs and to communicate with a network via the transceiver.

An aspect of the invention involves a wireless patient monitoring device including one or more sensors configured to obtain physiological information. The one or more sensors can include an optical sensor, an acoustic respiratory sensor, and/or a blood pressure measurement device. Other sensors, including, but not limited to, an EEG, ECG, and/or a sedation state sensor can also be used with the system of the present invention. The one or more sensors can be connected to a wireless monitor and configured to receive the sensor data and to wirelessly transmit sensor data or physiological parameters reflective of the sensor data to a bedside monitor/read-out. The bedside monitor can be configured to output the physiological parameters, communication channel, and/or communication status to the remainder of the system.

These and other features and advantages of the present invention will be better appreciated and understood when the following description is read in light of the accompany drawings.

### BRIEF DESCRIPTION OF THE DRAWINGS

The components in the figures are not necessarily to scale, emphasis instead being placed upon illustrating the principles of the invention. Moreover, in the figures, like referenced numerals designate corresponding parts throughout the different views.

FIG. 1 illustrates a schematic diagram of a patient alert and monitoring system of the present invention.

FIG. 2 illustrates a flow diagram of the patient alert and monitoring system of the present invention.

### DETAILED DESCRIPTION OF THE INVENTION

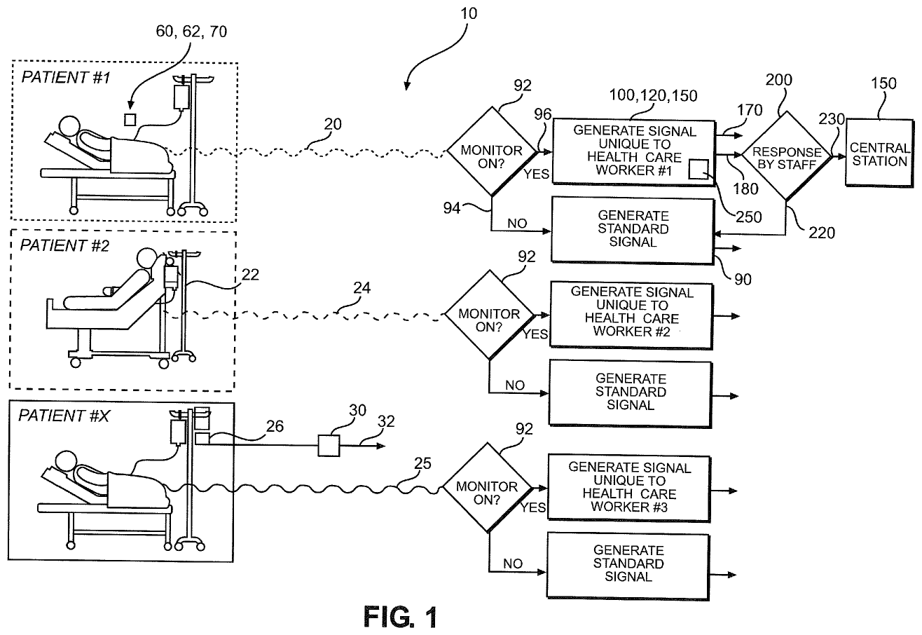

Referring to FIG. 1, the present invention pertains to a patient alert and monitoring system 10 for monitoring a plurality of patients, such as those indicated in FIG. 1 by Patient #1, Patient #2, and Patient #X. These patients may be located in a common room, such as an ICU or the like, where several patients are located, or they may be located in a private room, containing one or perhaps two patients. System 10 of the invention is also appropriate for use with various rooms, patients, areas, departments and health care workers. The patients, the rooms, the areas and/or the department are the responsibility of a particular staff member, such as a Registered Nurse, a team, and the like. Each patient may be connected to a health monitor, such as that indicated at reference numeral 26. These monitors 26 may be blood pressure monitors, oxygen monitors, heart monitors and/or other physiological indicators. The monitors, in general, are set for acceptable ranges.

Should a monitored vital sign or other characteristic move outside the acceptable range, action by hospital staff should be taken. In order to alert staff that action is required, the monitors 26 are connected to an alert system that is activated when the monitored data moves outside the acceptable range. The monitors 26 are connected to signal generators, such as signal generator 30, which generates a signal 32 when activated. The health monitors 26 may be connected to the signal generators wirelessly if desired.

In the system of the present invention, each patient or each set of patients for which a particular health care worker or team is responsible will have a unique signal. The signal is unique to a particular health care worker; therefore, other workers who may not be associated with the particular patient will not be distracted as they will understand that the alert/alert is unique to a particular health care worker who differs from them and the alert/alert is not their concern. For sake of convenience, the term “health care worker” will be used to represent nurses, doctors, teams, and other such members of a health care facility. Furthermore, the term “patient” will be shorthand for patients, areas of the facility, departments of the facility or the like. No limitation is intended by the use of these terms.

As shown in FIG. 1 and as discussed herein above, each patient and/or health care worker will have a unique signal, such as signals 20, 24, 25. Signal 20 can be an animal sound, such as a cat or a cow; signal 24 can be a dog or horse; and signal 25 can be a sheep; and the sound generated by these signals 20, 24 and 25 will be recognized by the particular health care worker responsible for the patient or group of patients. In this manner, a particular health care worker, for instance, a nurse, has a distinctive sound associated with him or her. All the patients for whom the nurse is responsible for will have that particular sound on their health care monitors. Therefore, when a health care monitor is activated, the unique sound will be generated and the health care worker will immediately know that it is her/his patient that is in need of assistance. Since the alert/alert sound is unique to one particular health care worker, the other health care workers will know that they are not being alerted, and, therefore, they will not be distracted from what they are doing at the time.

The signal generator is configured, upon initial power-up, to automatically transmit a signal to a local one of a plurality of networks via the transceiver to request the communications settings used by the local network, and to establish communications with at least one of the central stations on the local network using the communications settings received from the local network without intervention from a user of the patient monitor. The signal generated by the patient monitor will have been selected to be unique to a particular health care worker responsible for the patient, area, or department, whereby other health care workers will not be distracted into action. In some aspects of the invention, the wireless monitor can be configured to be coupled to an arm band attached to the patient. Alternatively, the wireless monitor can be configured to be coupled to a patient's belt, can be carried by the patient (e.g., via a shoulder strap or handle), or can be placed on the patient's bed next to the patient, among other locations.

In another embodiment of the invention, the wireless monitor includes a display screen. When the wireless monitor is within a pre-determined distance from the bedside monitor and transmits data over Bluetooth or Zigbee, a monitor display screen deactivates. When the wireless monitor is not within a pre-determined distance from the bedside monitor and transmits data over Wi-Fi or cellular telephony, the monitor display screen activates. Alternatively, independent of the communication protocol used by the device, when the wireless monitor is a pre-determined distance from the bedside monitor, the display screen activates. Similarly, in the alternative embodiment, when the wireless monitor is within a pre-determined distance to the bedside monitor, the display screen deactivates.

In certain embodiments of the invention, a blood pressure device can be used. The blood pressure device can be coupled to a medical patient and a wireless transceiver electrically coupled with the blood pressure device, such as a blood pressure cuff. The wireless transceiver can wirelessly transmit blood pressure data received by the blood pressure device and physiological data received from one or more physiological sensors coupled to the blood pressure device. To further increase patient mobility, in some embodiments, a single cable can be provided for connecting multiple different types of sensors together.

In still certain embodiments of the invention, the wireless patient monitoring device for measuring one or more parameters can measure oxygen saturation and respiration rate. The wireless monitoring device can connect to an oximeter probe and an acoustic respiration probe. The monitor can have a display screen and/or can transmit wireless information to a bedside monitor. In another embodiment of the invention, a docking station can be provided for the wireless monitoring device to dock it to a docking station forming a bedside monitor.

For the sake of convenience, the term “health care worker” will be used as shorthand for nurses, doctors, teams, and other such members of a health care facility. Furthermore, the term “patient” will be shorthand for patients, areas of the facility, departments of the facility. No limitation is intended by the use of these shorthand terms.

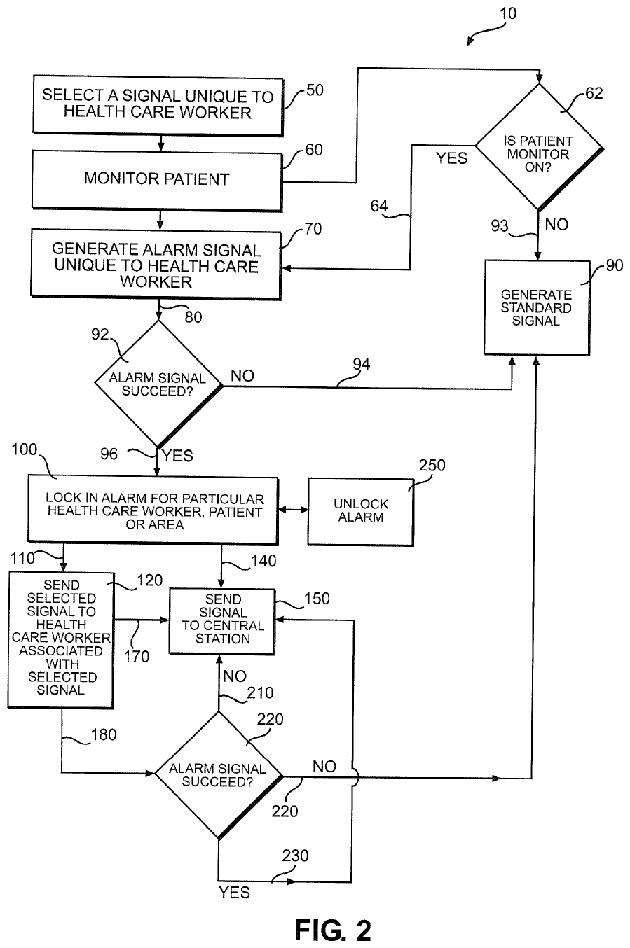

The patient alert and monitoring system 10 of the invention is more specifically demonstrated in the flow chart depicted in FIG. 2, and attention is directed thereto. Step 50 includes selecting a signal or sound that is unique to the patient or room and which is associated with a particular health care worker. Step 60 includes monitoring the patient for specific functions, such as blood pressure, heart rate, etc. The on/off state of the patient monitor is tested in step 62. If the patient monitor is “on” a go signal 64 is generated; if the patient monitor is “off” an “off” signal 93 is generated and sent to a standard signal generator 90. Step 70 includes generating an alert signal 80 if the monitored function moves out of a pre-set range. The distinctive and unique patient alert can be selected by operating control buttons on the alert generator.

In some embodiments of the invention, the alert generator may fail due to power or a malfunction or may be accidentally de-activated, for example. In a hospital setting, such failure may have serious consequences. Therefore, the patient alert system of the present invention preferably will have a fail-safe system which includes a signal generator 90 that generates a general signal to all personnel that all recognize and will be alerted accordingly.

In accordance with the above disclosures, the patient alert and monitoring system includes a step 92 that tests the viability of signal 80. If signal 80 is unsuccessful, a fail-safe signal 94 is generated in step 92 and sent to signal generator 90 to be sent out as a general alert to all staff. The general signal will be unchangeable and unavailable for use as a unique signal discussed above. The patient alert and monitoring system of the present invention also has step 60 generate a signal 93 if a patient monitor is turned off, which signal is sent to general signal generator 90 to be broadcast to all staff. On the other hand, if signal 80 is successful, a signal 96 is generated and locked in step 100 for the particular patient or room and/or a particular health care worker so other health care workers cannot select it for their use. A locked signal 110 is generated in step 100 and signal 110 is then used in step 120 to be sent to the pertinent health care worker as signal 180. In step 100, the locked signal is also sent as signal 140 to a central station 150, such as a central nursing station to be monitored and used as necessary. An alternative form of the system will include multiple computers as needed. It is noted that the patient alert system of the invention also has the ability to lock signals associated with certain rooms against use by any health care worker. Rooms such as a Trauma room or a Resus Room can be locked out. Such rooms can also have unique, unchangeable tones, and an alert tone if a monitor is off.

Once the pertinent health care worker has been notified in step 120, the identity of the pertinent health care worker is sent as signal 170 to the central station. The signal generated in step 120 can be received by a portable device carried by the health care worker, or by a circuit embedded in the worker's badge. If suitable, the signal generated in step 120 can be translated into colors for the worker. Various background/indicia combinations can also be used.

Once signal 180 has been sent to the health care worker, that worker can appropriately respond. However, should the worker fail to respond in a pre-set time, the system generates two signals 210 and 220, with signal 210 being sent to central monitor 150 to alert the monitoring entity that a response to alert signal 80 has not been attended to, and signal 220 is sent to standard signal generator 90 so that a general alert can be sent whereby all staff is alerted. Should the health care worker respond indicated in FIG. 1 at reference 200 (Response by Staff), a signal 230 is generated and sent to central monitor 150 so the general monitoring station is made aware that alert signal 90 is being attended to.

As is also indicated in FIG. 2, the system of the present invention also includes a way for a health care worker to lock an alert tone in a monitor when the worker signs out and thus avoids the need to reprogram an alert tone if the monitor is turned off. This feature is indicated as step 250.

Furthermore, the patient alert and monitoring system of the invention permits the display in central monitor 150 to display the name of a health care worker and an identifying symbol associated with the alert tone associated with that health care worker. The system can also be programmed so that alert signal 140 will also activate a flashing light next to the room or patient associated with signal 80.

The patient alert and monitoring system of the present invention differs from current day systems in that the alert from current day systems is coming from a patient monitor and sounding into a general area. However, in the patient alert system of the invention, the individualized signal sent from the patient is important because a health care provider is alerted to listen for a specific tone that relates that particular health care worker to their patient. This is an important feature if the health care provider has left the immediate area.

In some embodiments of the invention, wireless communication information can be stored on a data storage component and this facilitates communication between a wireless monitor and a bedside monitor. The information may be a unique identifier used to pair the wireless monitor with the bedside monitor. The information may be a password used to make sure only the correct receiver and only the designated health care provider have access to the transmitted physiological data. The information may be channel information to make certain the wireless monitor and bedside monitor communicate on the same channel.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims. Furthermore, while the present invention is described in the context of a hospital, it is noted that the invention can also be used in other settings, such as an elderly-care facility, and/or a retirement home without departing from the scope of the invention and claims presented herein.

What is claimed is:

1. A patient alert and monitoring system, comprising the steps of:
associating a particular health care worker with a patient;
assigning a distinctive and unique signal to the particular health care worker;
monitoring a selected physiological sign of the patient;
generating an alert signal when the selected physiological sign of the patient is out of a pre-set range; and
causing the alert signal to generate the distinctive and unique health care worker signal; and
sending the alert signal generated distinctive and unique health care worker signal to the particular health care worker.

2. The patient alert and monitoring system of claim 1, further comprising the step of generating a standard signal.

3. The patient alert and monitoring system of claim 1, further comprising the step of determining if the alert signal generated distinctive and unique worker signal is viable.

4. The patient alert and monitoring system of claim 1, further comprising the steps of providing a central monitor and sending the alert signal generated distinctive and unique health care worker signal to the central monitor.

5. The patient alert and monitoring system of claim 3, wherein the step of assigning a distinctive and unique signal to the health care worker includes selecting one of a plurality of sounds.

6. The patient alert and monitoring system of claim 4, further comprising the step of determining if the particular health care worker has responded to the alert generated distinctive and unique health care worker signal.

7. The patient alert and monitoring system of claim 6 further comprising the step of generating the standard signal if the particular health care worker fails to respond to the alert generated distinctive and unique health care worker signal.

8. The patient alert and monitoring system of claim 3, further comprising the step of providing a locked signal by locking the distinctive and unique signal associated with the particular heath care worker to the particular health care worker and locking out other health care workers from being associated with the locked signal.

9. The patient alert and monitoring system of claim 8, further comprising the step of locking the standard signal against use by any health care worker.

10. The patient alert and monitoring system of claim 4, further comprising the step of displaying the identities of the patient and the particular health care worker on the central monitor.

11. The patient alert and monitoring system of claim 10, further comprising the step of generating on the central monitor visual indicia associated with the alert generated distinctive and unique health care worker signal.

12. The patient alert and monitoring system of claim 2, further comprising the step of locking signals associated with selected rooms against being associated with any health care worker other than the particular health care worker associated with the assigned distinctive and unique signal.

13. The patient alert and monitoring system of claim 1, further comprising the step of generating a distinctive unique patient care signal if a patient monitor is turned off.
